(19) United States
(12) Patent Application Publication
(10) Pub. No.: US 2007/0116766 A1
(43) Pub. Date: May 24, 2007

(54) Gel compositions as muscle tissue simulant and related articles and methods

(76) Inventor: Darryl D. Amick, Albany, OR (US)

Correspondence Address: GANZ LAW, P.C., P O BOX 2200, HILLSBORO, OR, 97123, US

(21) Appl. No.: 11/452,660

(22) Filed: Jun. 13, 2006

Related U.S. Application Data
(60) Provisional application No. 60/692,391, filed on Jun. 13, 2005.
(60) Provisional application No. 60/751,183, filed on Dec. 16, 2005.
(60) Provisional application No. 60/788,680, filed on Apr. 3, 2006.

(51) Int. Cl. A61K 9/14; C08K 5/00

(52) U.S. Cl. 424/486; 524/515

(57) ABSTRACT

Gel composition comprising thermoplastic block copolymer and oil suitable for use as simulants for body tissue, and methods related to the compositions.

### RELATED APPLICATIONS

This application claims the benefit of and priority to U.S. Provisional Application Ser. No. 60/692,391, filed Jun. 13, 2005, U.S. Provisional Application Ser. No. 60/751,183, filed Dec. 16, 2005; and U.S. Provisional Application Ser. No. 60/788,680, filed Apr. 3, 2006, the contents of which are hereby incorporated by reference as if recited in full herein for all purposes.

### BACKGROUND

The inventive subject matter disclosed herein relates generally to a composition useful for simulating mammalian muscle tissue in a variety of applications. In certain applications the composition is an improved ordnance gel for use in ballistic testing and studies.

Traditionally, various grades and types of gelatin, an animal derivative comprised primarily of a mixture of certain animal proteins colloidally dispersed in water, have been used to simulate mammalian muscle tissue. Gelatin is widely accepted and is the only material approved and used by various government agencies as an ordnance or ballistics gel to evaluate the traumatic effects of bullets, shrapnel, and other projectiles on muscle tissue. It has also been used to estimate potential trauma in humans produced by explosions, auto accidents, and other trauma inducing events.

Unfortunately, there are serious disadvantages to the preparation and use of gelatin that make it an inconvenient and expensive composition to use. The ingredients and manner in which gelatin is prepared readily illustrate how problematic it is.

A typical ordnance gel composition of 10 wt % gelatin solution in water has been shown to simulate swine muscle—but only when cooled to, and maintained at about 4° C. (39.2° F.). Accordingly, the preparation and use of the gelatin requires cooling and refrigeration equipment not only for preparation but also for testing. In some instances, this has meant that law enforcement agencies have had to furnish cooled trailers for carrying-out testing in the field. If such temperature cannot be maintained in the lab or field where the gelatin is to be used, the gelatin must be used quickly, usually within about 20 minutes before it warms to an unacceptable temperature.

Another problem with gelatin is that its composition is complex and variable because (1) it is derived from animal by-products and (2) it has a propensity to lose or gain moisture. Therefore its material properties may vary significantly, both within a monolithic body and from batch-to-batch.

Still another problem is its limited shelf life. The organic and aqueous makeup of gelatin makes it an ideal breeding ground for bacteria, fungi, mold and other microbes, some of which may be pathogens. This characteristic results in progressive decomposition, odor, unsanitary conditions, etc. So not only must the gelatin be cooled to maintain it with appropriate material properties for testing, but it also must be cooled to maintain its shelf life. Spoiled gelatin may also pose a disposal problem in that it may be classified as a biohazard or pollutant, especially when prepared in bulk.

Any number of other problems or disadvantages may arise from the manner in which gelatin must be prepared and delay before it is ready for use. For example, gelatin composition and material properties may be adversely affected by straying from temperature, dissolution, agitation, and gel setting parameters.

Another problem with gelatin is that it is not readily reusable once it is prepared. Therefore, new batches of gelatin are prepared to replace those used in testing.

Some of the other difficulties encountered in the use of gelatin may be exemplified by considering the evaluation of an ordnance gelatin block which has been shot with a projectile, such as a pistol or rifle bullet to determine its effect upon impact with tissue. In passing through the gelatin, a relatively large “temporary cavity” is created which envelopes the projectile. Some part of this cavity acts in an elastic manner and instantaneously contracts back toward the center of the bullet path as the bullet or bullet fragments departs from any given planar region perpendicular to the bullet trajectory. The portion of the temporary cavity which deforms inelastically (i.e., plastically) remains as the so-called “permanent cavity”, which may be surrounded along some portion of its length by radial fractures associated with the former temporary cavity. If the gelatin has been prepared properly and is of sufficient clarity, these time-dependent events may be recorded by the use of high-speed photography (e.g., at least 10,000 frames/second). However, most often a ballistics technician is assigned to cut the gelatin block into slices perpendicular to the bullet path and to tediously document the lengths and numbers of radial fractures at various planes.

Another important task of the ballistician may be to identify and retrieve bullet fragments from the gelatin for further evaluation of the bullet's behavior. Both of these tasks are labor-intensive and tedious, and performed under the unpleasant conditions inherent in the nature of gelatin, such as the handling of sticky, spoiling, putrefying gelatin. It should be understood that the evaluations performed on ordnance gelatin blocks after shooting, whether done photographically or manually/optically, depend upon acceptable gelatin clarity. The clearer the gelatin medium, the greater the opportunity to witness and record details of bullet behavior. Unfortunately, gelatin may not be sufficiently transparent to allow resolution of all relevant detail from a ballistics test.

While there have been attempts to develop, produce, and market non-gelatin muscle tissue simulants, based, for example, on glycerin soaps, animal tallow derivatives, waxes, clays, and putties, and polymer gel candle compositions, all suffered from one or more serious problems or disadvantages in terms of preparation, performance and use. Some problems include insufficient rigidity or elasticity to simulate muscle tissue. Others include insufficient transparency to resolve projectile-impact details, non-recyclability, or susceptibility to moisture.

In view of the aforementioned problems and disadvantages, there is great need for improved muscle simulants that may be used in ballistics applications and in other applications requiring a facsimile of muscle tissue, and which have one or more of the following needed attributes:
1. Simulates muscle tissue, at least per ballistic testing standards;
2. Relatively easy to prepare;
3. Usable and preserved at typical room or field temperatures without controlled cooling and has long shelf life;
4. Can be made transparent to facilitate viewing of projectile impacts and effects;
5. Resists microbial growth;
6. Has homogeneity;
7. Is reusable or disposable in an ordinary manner;
8. Allows for integration of other anatomical simulants, such as bone and organs;
9. Is not water-based and subject to water-related problems, such as variance in material properties induced by environmental conditions (e.g., humidity);
10. Allows projectile pieces to be easily retrieved or removed;
11. Provides a synthetic muscle-tissue simulants (“SMTS”) for use as a ballistic medium which accurately records the “temporary cavity” by high-speed photography and which preserves the “permanent cavity” for later evaluation;
12. Provides an SMTS that does not exhibit fracturing in the region of the “temporary cavity”, and which is therefore capable of recording a greater number of rifle or pistol shots than would be possible with ordnance gelatin;
13. Provides an SMTS that may be handled and stored without significantly degrading surface quality (i.e., smoothness, gloss etc.);
14. Suitable for education and training: for example, medical technologists (for example, surgical students) with an SMTS artificial organ or other shaped body suitable for teaching, practicing, etc., and such articles may also be used in the evaluation of various surgical instruments, devices, etc.
15. Suitable for use as a prosthetic device, properties of which may be tailored to closely simulate various parts of the human body; and
16. Provide forensics and trauma technicians with an SMTS by which to study, not only wounds (ala bullet trauma), but also other types of damage to the human body, such as impact, shock, energy absorption, etc.

### SUMMARY

The inventive subject matter disclosed herein overcomes the foregoing problems and satisfies the foregoing needs by generally providing certain novel compositions and related methods of making and using the compositions. In certain possible embodiments, the inventive subject matter contemplates the following:

A non-aqueous gel composition that may comprise thermoplastic block copolymers and an oil, the block copolymer being in a concentration that imparts to the gel composition sufficient properties to pass the FBI ordnance gel protocol when the gel composition is about 50-80° F. The gel composition may be substantially transparent. The gel composition may include a material or structure for simulating a body part as a discontinuous phase of the gel composition. The block copolymer comprising the gel composition may be selected from the group of polymers comprising (i) styrene-butadiene-styrene polymers; (ii) styrene-isoprene-styrene polymers; (iii) styrene-ethylene-butylene-styrene polymers; (iv) styrene-ethylenepropylene polymers; (v) styrene-ethylenebutylene polymers; (vi) styrene-butadiene polymers; and (vii) styrene-isoprene polymers. The gel composition may include about 12 wt % to about 22 wt % of the block copolymer. The block copolymer may comprise a styrenic block copolymer. The styrenic block copolymer may be a hydrogenated styrenic block copolymer. The gel composition may be configured for use as a protective pad or comfort cushion.

A kit comprising a heating unit and a gel composition or the raw materials for a gel composition, the heating unit providing a receptacle area for melting the materials or gel composition to a predetermined temperature and forming a body of gel. The heating unit of the kit may be sized and shaped to provide a body of gel of at least 2″×2″ cross-section.

A method of making a muscle simulant comprising combining a thermoplastic block copolymer with an oil, applying heat so as to heat the mixture to about 200° F. to about 260° F., allowing the combination of materials to cool into a gel composition, the composition being formed anhydrously. The block copolymer may be selected from the group of polymers comprising: (i) styrene-butadiene-styrene polymers; (ii) styrene-isoprene-styrene polymers; (iii) styrene-ethylene-butylene-styrene polymers; (iv) styrene-ethylenepropylene polymers; (v) styrene-ethylenebutylene polymers; (vi) styrene-butadiene polymers; and (vii) styrene-isoprene polymers. The composition may include about 12 wt % to about 22 wt % of the block copolymer. The copolymer may comprise a styrenic block copolymer. The styrenic block copolymer may be a hydrogenated styrenic block copolymer. The method may allow the gel to form under conditions that prevent the formation of appreciable bubbles. The method may add a material or structure for simulating a body part as a discontinuous phase of the gel composition. The method may comprise shaping the gel composition so that it is suitable for use in an FBI ordnance gel protocol. The method may comprise shaping the gel composition so that it replicates a body part.

A method comprising providing a non-aqueous gel composition comprising a styrenic block copolymer and an oil, the gel composition adapted to pass the FBI ordnance gel protocol, and using the gel composition for ballistics testing. The method may comprise melting the gel composition after testing and allowing it to reform as a gel in a desired shape.

A method comprising providing a gel composition comprising a styrenic block copolymer and an oil and forming the gel composition into a shape that replicates a body part. The method may comprise providing a nonaqueous gel composition comprising a styrenic block copolymer and an oil, the composition having a predetermined shape for simulating a body part, and using the shaped gel in an educational or medical training program or study unrelated to ballistics. The method may comprise forming a discontinuous phase in the gel. The method ma)y comprise forming a functional gradient in the gel. The discontinuous phase may correspond to a body part or region. The functional gradient may correspond to a body part or region. The gel composition may be configured for use as a protective pad or comfort cushion.

A gel composition comprising a thermoplastic block copolymer and an oil wherein the composition is configured for use as a comfort or protective pad; a prosthetic device; or a diving weight. A discontinuous phase may be present in the gel.

These and other embodiments are described in more detail in the following detailed descriptions and the figures. The foregoing is not intended to be an exhaustive list of embodiments and features of the present inventive concept. Persons skilled in the art are capable of appreciating other embodiments and features from the following detailed description in conjunction with the drawings.

### BRIEF DESCRIPTION OF THE FIGURES

FIG. 1 represents a kit comprising a body of a gel composition and a heating unit for melting or forming the composition, according to the inventive subject matter.

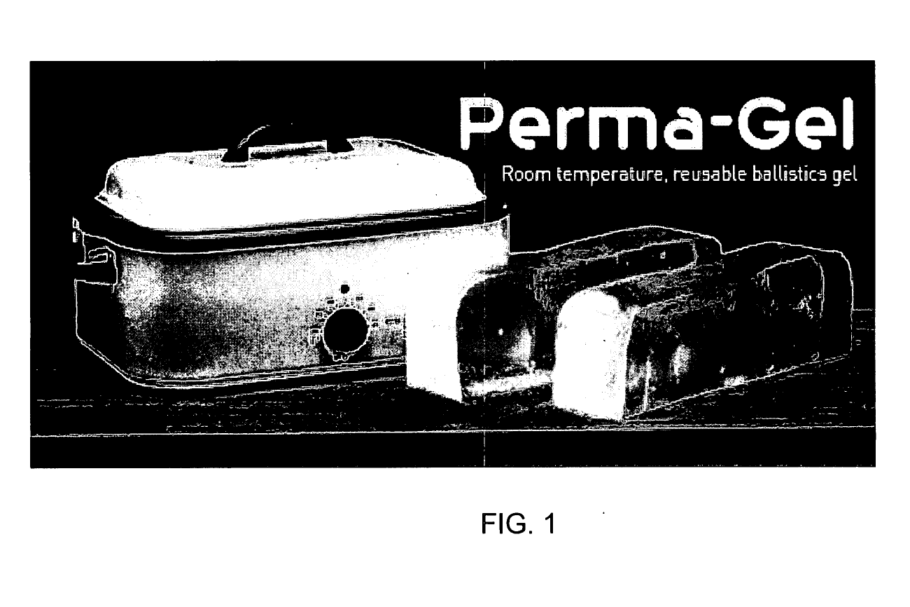

FIG. 2 represents a block of gel used in an ordnance test, with viewable projectile tracts, cavities and pieces, according to the inventive subject matter.

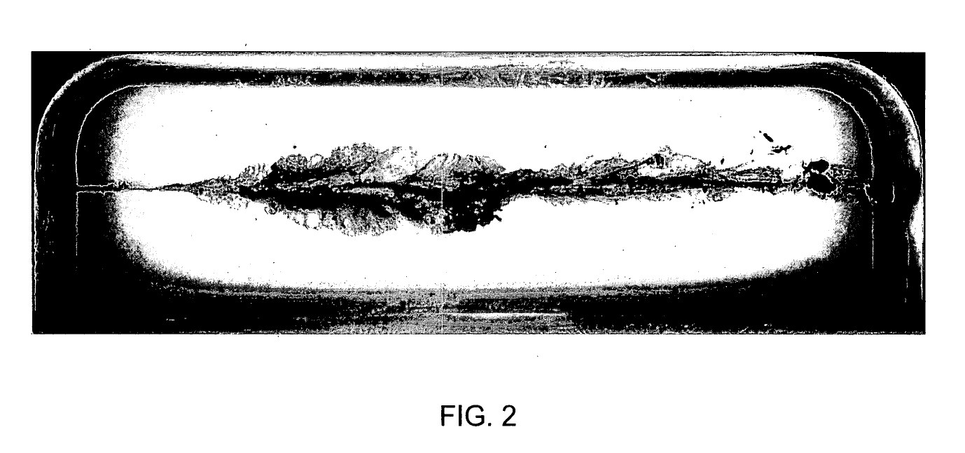

FIG. 3 represents a body of gel including simulated body parts, the parts being transparent and forming a discontinuous phase in the gel, according to the inventive subject matter.

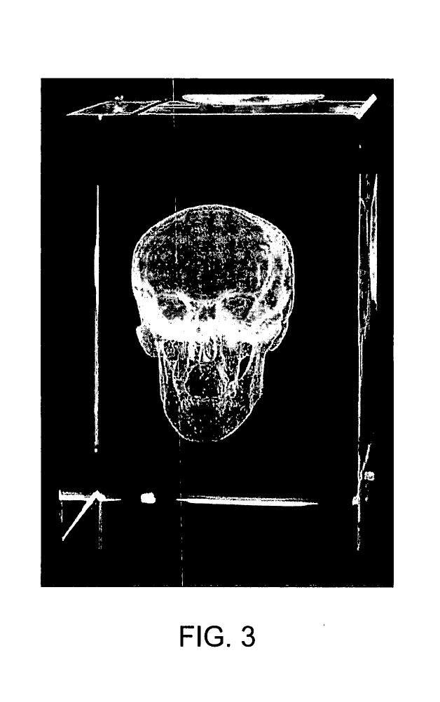

### DETAILED DESCRIPTION

It has been unexpectedly found that admixing certain thermoplastic copolymers in certain oils (e.g., hydrocarbon oils, such as white oil or mineral oil) under controlled conditions of temperature, time and moisture content results in gel compositions which, for a variety of applications, simulate muscle tissue or provide viscoelastic properties for other applications. The gel compositions according to the inventive subject matter disclosed herein are particularly suitable as ballistic gel to replace current ordnance gelatin. In fact, gel compositions according to the inventive principles may be superior to traditional ordnance and medical gels in many respects including clarity, chemical stability, waterproofness, anti-bacterial properties, reusability, disposability, handleability, safety, and economics.

Gel compositions according to the inventive principles may be formed from thermoplastic block copolymers and an oil, yielding a gelatinous body of material (also referred to herein as a “gel” or “gel composistions”) that may be characterized as elastomers or gel elastomers (viscoelastomers). Block copolymers are generally composed of sequences of the same monomer unit as one block-type, covalently bound to unlike sequences as another block-type. The blocks can be connected in a variety of ways. The different blocks can sometimes intermix freely at sufficiently high temperature, or when sufficiently diluted with solvent, generating a more disordered form. However, it is common for the blocks to spontaneously self-assemble (“order”) into a diversity of mesophases, with the size scale governed by the chain dimensions (order may be tens of nanometers). In the mesophases, dissimilar (e.g., thermodynamic dissimilarity) blocks exist in distinct “microdomains” which are highly enriched in blocks of the same type, sometimes to the point of being essentially pure. The covalent bonds linking the dissimilar blocks are thus localized to the vicinity of the microdomain interfaces. The block ratio is easily varied during polymer synthesis to alter the mesophase structure. The known equilibrium mesophases for diblock copolymers include spheres, cylinders, gyroid, and lamellae.

As used herein “block copolymer” is not limited to di-blocks and may encompass triblocks, tetrablocks, pentablocks, etc. Block copolymers may be made, for example, using “living polymerization” techniques, such as atom transfer free radical polymerization (ATRP), reversible addition fragmentation chain transfer (RAFT), living cationic or living anionic polymerizations.

Particularly suitable polymers for use in the inventive compositions are thermoplastic block co-polymers that are based on at least two relatively thermodynamically incompatible or dissimilar block segments. Many kinds of styrenic block copolymers are expected to be suitable. Hydrogenated styrenic block copolymers are believed to be particularly suitable. The following block copolymers are specifically contemplated: styrene-butadiene-styrene polymers; styrene-isoprene-styrene polymers; styrene-ethylene-butylene-styrene polymers; styrene-ethylenepropylene polymers; styrene-ethylenebutylene polymers; styrene-butadiene polymers; and styrene-isoprene polymers. One or more of the blocks may be a random copolymer. Suitable copolymers are sold under the name SEPTON™. These co-polymers are a series of thermoplastics and are available from KURARAY CO., LTD. and via related company Septon Company of America, Pasadena. Tex. (http://www.septon.info/en/septon/what_septon.html). In terms of structure, the SEPTON™ series polymers generally are a series of hydrogenated styrenic block copolymers that exhibit rubber-like properties. More specifically, several types of hydrogenated styrenic block copolymers of SEPTON™ include a hydrogenated poly(styrene-b-isoprene)(SEP), a hydrogenated poly(styrene-b-isoprene-b-styrene) (SEPS), a hydrogenated poly(styrene-b-butadiene-b-styrene)(SEBS) and a hydrogenated poly(styrene-b-isoprenelbutadiene-b-styrene)(SEEPS).

As persons skilled in the art will recognize from the teachings herein, these block copolymers, can be chemically modified with various molecular and atomic constituents to alter the desired chemical or physical property of the gel. In addition, the monomeric groups in a block can be substituted with an alternative monomer providing differential performance from the original copolymer used. For example, it is expected that styrene may be replaced with other aromatic monomers such as naphthalene, and butadiene could be replaced with an alternative alkene. All such modifications or derivatives are intended to be within the scope of equivalency.

Suitable oils in which the block copolymers may be mixed with or dispersed in include: mineral oils and other hydrocarbon oils; organic oils (vegetable and animal based), synthetic oils similar to any of the foregoing, and mixtures thereof.

In certain embodiments, muscle simulants are prepared by dissolving about 12 wt % to about 22 wt % of at least one thermoplastic block copolymer in oil (e.g., specific types of hydrocarbon oils) under prescribed degrees of temperature control (e.g. from above 230 to below 300° F.), by limiting moisture (i.e., water) content to a maximum of no more than about 100 ppm, by taking precautions to avoid forming and entrapping appreciable gas bubbles (including, but not limited to, air) and by using certain measures described herein in combining polymers with oil to avoid the formation of high-polymer “clots.”

Certain similar gel compositions (and methods of manufacture) for use in candle making are described in U.S. Pat. Nos. 6,066,329; 5,879,694; and 5,578,089, which are hereby incorporated by reference as if set forth in their entireties. These patents do not disclose or suggest that the gels are suitable for use as a muscle simulant or as an ordnance gel. Certain gels that are believed to be made according to the teachings of one or more of the foregoing gel candle patents were tested and found to be insufficient to meet an ordnance gel test promulgated by the FBI and/or suffered other drawbacks that render them unsuitable for use as improved ordnance gels or muscle simulants. However, the above-named inventor has unexpectedly discovered that the general compositions disclosed in the patents, can be adapted into improved gel compositions that meet the FBI ordnance gel test protocol or may otherwise be made superior for use as a muscle simulant, as indicated in more detail below.

Ordnance Gelatin Calibration

According to FBI standards, calibration of ballistic gelatin or gel composition is verified by firing a .177 steel BB at 590 feet per second (fps), plus or minus 15 fps, into a block of gelatin or synthetic gel composition resulting in 8.5 centimeters (cm), plus or minus 1 cm, penetration (2.95″−3.74″). For handgun rounds, the gelatin is formed in a block of about 6″ square and 16″ long. During FBI tests, any gelatin blocks which fail the calibration test are discarded. In the case of gelatin, the gelatin is taken from refrigeration at 39.2° F. and used within 20 minutes of being taken from the refrigerated condition. (Hereinafter, these test parameters may be referred to as the FBI ordnance gel standard or protocol.)

In addition to the specific requirement of a muscle simulant to meet specified ranges of rigidity, elasticity, penetrability and clarity, ballistic media should not include any appreciable bubbles because of their effect on bullet penetration, “mushrooming”, yawing, etc.

An advantage of the gel composition disclosed herein is that they are usable at around room temperature and are thermally reversible so that they may be remelted and reused. As used herein around or about room temperature means about 50-80° F.

Further, the inventive subject matter includes a complete recycling kit for use in the home or laboratory is presented, comprising one or more of an initially-supplied SMTS bodies, an appliance with appropriate temperature control, one or more molds and, optionally, one or more types of filters by which molten SMTS may be cleaned and scavenged of fragments for reuse. The details of contemplated kits are described in more detail below.

While the foregoing teachings on gel composition are believed to provide a variety of suitable SMTS gels, the following are examples of both unsuccessful and successful attempts to make acceptable SMTS bodies. These are intended as guide posts to help those skilled in the art arrive at suitable gel compositions. The Examples herein are believed to be accurate and reproducible. However, the Examples are intended to supplement the foregoing teachings, and, as is always the case with any experiment, may not have been carried out in a manner that is perfectly in accordance with scientific principles or otherwise beyond question. Accordingly, the following Examples should be viewed as prophetic in nature, but the Applicant reserves the right to rely on them as actual experiments should it be necessary to overcome certain kinds of rejections that might arise during the examination of this patent document.

Unless otherwise indicated, methods of preparing the formulations in the Examples are provided under the section below, “Process Conditions.”

### EXAMPLE 1

One suitable composition according to the inventive subject matter is prepared from about 16.7 wt % Septon 4033, one of the SEPTON series copolymers described above, plus 83.3 wt % white oil. The white oils may be Superla 7 available from Chevron Products Co., San Ramon, Calif. or STE 70, available from STE Oil Company, Inc. San Marco Tex., both of which have about the same viscosity. A similar polymer is Kraton G1650, which is a clear, linear tri-block SEBS copolymer.

### EXAMPLE 2

Another suitable embodiment contemplates dissolving 14-18 wt % Kraton G-1650 in a white mineral oil (e.g., Chevron Superla 7, Chevron Products Co., San Ramon, Calif.). This formulation produces an SMTS which, at room temperatures (e.g., 55-75° F.), essentially matches the ballistic penetration resistance of chilled (4° C./39.2° F.) 10% ordnance gelatin. Kraton series polymers are supplied by Kraton Polymers, US, LLC, Houston, Tex.

### EXAMPLE 3

Three varieties of Versagel C, a Penreco Versagel series gel made for candle-making, were melted at about 225° F. in a mold of approximate dimensions 8-in. dia.×6 in. long and allowed to solidify. Molded blocks were then shot with a BB gun (177-in. dia. steel) at about 600 ft/sec.(fps) at a block temperature of about 4° C. (39.2° F.). The Versagel series copolymers are available from Penreco, The Woodlands, TX. Results for the three available candle grades (“low-polymer”, “medium polymer” and “high-polymer”) were as follows:
LP: greater than 6 inches
MP: greater than 6 inches
HP: about 6 inches
   All three available gel-wax candle Versagels failed to meet the FBI requirement for 7.5 cm (2.95 in.)−9.5 cm (3.74 in.) penetration in ordnance gelatin under the conditions given.

### EXAMPLE 4

A special “extra-high-polymer” grade of Versagel (CXHP) candle wax was obtained from Penreco and subjected to the same testing as described in EXAMPLE 3. Resulting penetration at 4° C. was about 4½ inches.

### EXAMPLE 5

Two additional varieties of proprietary gel-wax candle compositions (identified as “106” and “107” Versagel) were supplied by Penreco in further attempts to identify a material with acceptable penetration-resistance. The 106 gel exhibited excessive penetration (about 4¼ in. at 4° C.) and was rejected. While the 107 composition exhibited acceptable penetration (about 3¼ in at 4° C.), an unexpected problem was noted. This gel-wax, at the subject cool temperature, was so opaque that contained projectiles (i.e., BB's) were not visible. Upon further investigation, it was found that the transition from “transparent” to “opaque” occurred at about 47° F. above which penetration was excessive. This behavior was deemed to eliminate the usefulness of Versagel 107 as an acceptable ballistic medium.

### EXAMPLE 6

In view of the failures described above, further compositions were prepared that were hoped not only to satisfy FBI ordnance gel protocol of 4° C. (39.2° F.), but perhaps even at room temperature. Such a material would offer significant improvements to ballisticians, including potentially significant cost savings. A series of compositions were evaluated by preparing small “muffins” (about 2″dia.×¾″-thick) of different combinations of oils, a block copolymer (Kraton G-1702) and a block copolymer (Kraton G-1650). Oils evaluated included vegetable oil (soya bean), new hydraulic oil (AW-46 from three different refineries), used hydraulic oil (unfiltered, unreprocessed AW-46) and white mineral oil (three different grades).

The following is a brief summary of findings from the various experiments:
1) Compositions including block copolymer Kraton G-1702 produced materials which were low in rigidity, difficult to handle due to stickiness or susceptibility to surface damage.
2) Copolymers dissolved in vegetable oil (e.g., soya bean oil) produced materials which were quite opaque at room temperature, and therefore unacceptable for ballistics testing, but possibly acceptable for many other applications where transparency is not required.
3) Block copolymer Kraton G-1650 dissolved in hydraulic oil in the range of about 12.3 wt % to about 12.7 wt % polymer produced materials that had acceptable penetration resistance (2.95-3.74 inches; 600 fps, BB) at 4° C. (39.2° F.).
4) A concentration of 15 wt % of a Kraton polymer in Chevron ISO 2000 AW 46 hydraulic oil produced a material which had acceptable penetration at room temperatures between 51° F. and 82° F. This material was light yellow in color and had an unpleasant odor, probably due to chemical additives for corrosion inhibition. Other brands of AW 46 oil (SouthCorp and Exxon) had darker colors and even stronger odors, although materials made therefrom also had acceptable rigidity. Used AW 46 oil (brand unknown) produced very dark materials and strong odors. (New and used hydraulic and other oils are, nevertheless, of interest because the), are widely available products at lower or zero cost.)
5) Chevron white mineral oil “Superla 7” was mixed with 15 wt %-17.4 wt % Kraton G-1650 to produce a family of materials with acceptable penetration resistance at room temperatures between 50° F. and 80° F.
   These materials were deemed to be quite satisfactory in that rigidity, color, clarity, odor, resistance to handling, etc., were all within acceptable ranges.
6) Using 16 % Kraton G-1650 polymer in Superla 7 oil, it was observed that a significant degree of variability in properties was intermittently encountered. Not only did rigidity vary, but such properties as “tackiness”. color and odor during heating were also observed to vary unexplainably. It is believed that these variances were all due to excessive temperatures during dissolution of polymer into the oil. Specifically, it was determined that temperatures above 260° F. and particularly those above 275° F. caused the mixture to be degraded, as evidenced by the presence of light concentrations of smoke, the generation of gas bubbles, slight darkening and the tendency of the finished material to become somewhat tacky/sticky. This finding was surprising, because the flashpoint of Superla 7 (360° F.) is comfortably above the temperatures found to result in degradation.

It was also found that substantial care should be taken in selecting the appliances and methods used in dissolving Kraton G-1650 in Superla 7. One is faced with the requirement of heating the polymer/oil mixtures to temperatures sufficiently high to reduce viscosity and thereby allow bubbles to rise and escape from the surface, while avoiding any thermal degradation. It is very easy to inadvertently scorch localized regions in the mixture, due to encountering localized temperatures in excess of 260°-275° F. The difficulty in avoiding this can be appreciated by considering that a conventional electrical heating element operates at a surface temperature of about 900-950° F. Attempts to use a large commercial baking oven resulted in smoking/scorching, even when, for example, top elements were disconnected. Because of this problem, it is proposed that acceptable means of heating polymer/oil mixtures should be confined to indirect methods such as convection ovens, double-vessel designs (e.g., “double-boilers”), oil-in-tube heaters, steam heaters, and other heating units, et al.

Another significant finding was that the presence of even traces of moisture may result in bubble formation. This is because water has a density higher than that of polymer/oil mixtures and therefore sinks to the bottom of the melting vessel as discrete, immiscible droplets. When the temperature of the mixture reaches about 212° F., steam bubbles begin to form. But this takes place over a considerable length of time, so that bubbles continue to rise toward the surface throughout the finishing stage of the melt cycle. Even one drop of water or sweat can ruin the integrity of an entire batch (e.g., several gallons) of material, rendering it unacceptable for use under the FBI ordnance gel protocol; but possibly acceptable for many other applications, such as an SMTS in medical education and training.

In combining and dissolving the relatively high concentrations of Kraton G-1650 polymer in oil, some attempts were made to accelerate the process by preheating the oil to about 225° F. and then slowly adding polymer. It was observed that this abrupt contact between polymer and hot oil caused very high-polymer (and therefore high melting point) clots to form. This created hard spots, as well as bubble entrapment, conditions which were very difficult to remedy by excessively long holding times at temperatures above about 225° F. A solution to this problem was found by pre-blending at room temperature to elevated room temperature (i.e., less than 120° F.) polymer and oil, ensuring that complete intermixing occurs, and then heating to above 200° F. to obtain gelling. These preblended mixtures may be heated either in bulk or by making incremental additions of the same to an already-melted pool.

Techniques were developed for producing full-size ballistic blocks (e.g., 6×6×16-in. or 8×8×20-in.) and a variety of other shapes (e.g., artificial eyes and other organs), both by melting directly in a mold or by first melting in a vessel and then pouring into a mold. Techniques for preventing bubbles, including the use of vibration and/or vacuum.

Process Conditions

The following illustrates a set of representative steps and conditions for manufacturing a gel composition, according to the inventive subject matter disclosed herein. The specific ingredients are representative and the process may generally be used with other formulations.
1) Blend 4.5 lb Septon 4033 with 3.18 gal. (22.42 lb, 7.05 lb/gal) Chevron Paralux 1001R Base Oil in a standard cement mixer for about 10-15 minutes, or until all visible polymer lumps are dispersed.
2) Pour into suitable containers, such as the inner pan of an “oven kit” or some other type of oil-resistant container, and seal. (See details below.)
3) Before applying heat, allow at least about 12 hours when ambient air is above about 60° F. or at least about 24 hours at cooler temperatures. Failure to allow sufficient time may result in bubble formation, as discussed below.
4) During all operations, including storage of raw materials, do not allow any appreciable amount of water (e.g., human sweat) to contaminate the product or ingredients. (Severe bubble formation during melting may result from the presence of water.)

During the 12-24 hr dwell time referred to above, several important things should occur so that the gel is in good order for use and/or shipment. First, in order to ship the raw, unmelted/ungelled mixture, it must set or gel enough that it will not be prone to spill. (Before this occurs, the consistency of the mixture is rather like oatmeal mush.) Once the mixture has gelled, consistency is about like a weak foam rubber, in that it will remain in an inverted container without falling out. By way of example, after a few days at normal room temperatures, rigidity of the “foam rubber” has increased to a degree that it may be necessary to tear or cut it into pieces in order to remove the block of mixture from its container. Packaging of the oil-based gel composition materials should include some type of an oil diffusion barrier, such as polypropylene or high-density polyethylene. For wrapping finished gel blocks, certain types of silicone-treated paper work well and do not tend to stick to the gel surface as most other substances do.

In contrast to commercial candle gel compositions, ballistics gel compositions need to use higher polymer concentrations to have gel stiffness/strength sufficiently high to meet the normal BB penetration test. In departing from gel compositions actually used for gel candles, there are several problems solved according to inventive principles disclosed herein. First, the higher polymer loads require higher temperatures to be used for melting. At typical candle-based temperatures (generally less than 200° F.), viscosities are so high as to inhibit the rate at which entrained air bubbles can rise to the surface of the melt. Raising the temperature to lower viscosity is a partial solution to this problem, but temperatures in excess of 260-270° F. result in rapid oxidation of the oil.

For the polymer-oil mixtures disclosed herein, melting temperatures in the range 225-260° F. are satisfactory. When inadequate “dwell” times (i.e., soaking time of polymer in oil) are used prior to the application of external heating, a very undesirable phenomenon was observed. If one pictures polymers as “curds”, some of which are as large as ¼-inch in diameter, it is necessary to allow oil to thoroughly penetrate and disperse such particles by diffusion before attempting to heat the mixture to form homogeneous gel. If gelling begins to occur at the surface of a curd which still contains entrapped air at its core. a very high-polymer “skin” forms and envelopes polymer-air mixtures at or near the core. This condition not only creates entrained air (and its potential for forming bubbles), but it is also found that the high-polymer “skin”, with its inherently high melting point/viscosity relationship is nearly impossible to dissolve without scorching the mixture (i.e., without oxidizing the oil). As with essentially all heating operations in which oxidation is an undesirable consequence, both time and temperature contribute to oxidation. For this reason, any factor which causes unnecessary increases in either parameter should be avoided, or at least minimized.

In contrast, it is believed that candle formulations tend to favor high viscosities, because of their inherently higher flashpoints, but SMTS gels prefer lower viscosities which accelerate the interaction between polymer and oil. (In fact, this is one more reason that a co-polymer such as Septon 4033 is preferred over Kraton 1650: it has a high affinity for oil.) A high-viscosity oil (Citgo DuroPreme 200) has been used, but it has been found that it added about 20-25% to the normal compounding/melting cycle time. It also had an effect on gel rigidity, in that the polymer concentration had to be reduced to 93% of the value needed for less-viscous (70) oil. In ballistic medium applications, primary emphasis is placed on minimizing the frequency and size of bubbles (because of their potential effects on bullet performance) and on avoiding the degradation of gel physical/mechanical properties. These requirements are not the same as those important to candle applications.

As noted, a high-polymer skin can form around the polymer curds, this same problem may also occur when speeding up the melting cycle by preheating the oil before adding polymer to it. Accordingly, the gel should not be formed at a rate which is too rapid. In other words, the gel reaction rate is probably diffusion-limited, rather than determined by thermal factors (heat input rate, thermal conductivity, thermal capacity/specific heat, etc.). This analysis is further supported by observations that constant agitation, shearing, comminution of the unmelted polymer-oil mass (e.g., by chopping or cutting), etc. seem to have beneficial effects on reducing the melt cycle time. It is noted that whereas the initial melt of virgin raw materials takes about 5-6 hours to produce a 27-lb melt in the roaster oven, remelting the gel (e.g., after shooting) requires only 3-4 hours.

In summary, suitable formulations and methods for producing an acceptable synthetic muscle tissue simulant (SMTS) include dissolving about 12 wt % to about 22 wt % of at least one thermoplastic block copolymer into an oil, such as a hydrocarbon oil, at sufficiently high process temperatures sufficient to dissolve the mixture and to lower viscosity sufficiently so that bubbles of gas, steam, air, et al may escape to the surface before solidification is obtained. A suitable temperature range may be from about 200° F. to about 300° F. for most gel compositions. A more suitable temperature may be between 230° F. and 275° F., and even more suitable range may be between 230-250° F.

Prescribed precautions should generally be taken to help achieve a body gel having desired properties and performance and the following more specific parameters may be followed:
1) Even localized “hot spots” exceeding 275° F. (and preferably 260° F.) must be avoided. The heating methods and appliances selected should conform to this specification.
2) Moisture (i.e., water) must be eliminated or sufficiently minimized so that it does not become entrained in the polymer/oil mixture. This includes, but is not limited to, perspiration from workers.
3) For optical clarity, such as that required for ballistics photography, at least one glossy viewing surface should be provided on the material body. This may be accomplished by using such materials as glass, ceramic, polished metal, etc as a side of the mold.
4) During initial melting or remelting, sufficient time at the appropriate temperatures should be allowed in order for bubbles of air, steam, decomposition products and volatile impurities to escape.
5) Contact between hot oil (i.e., above 120° F.) and raw polymer should be avoided, in order to prevent the formation of high-polymer clots and entrapped bubbles.

The finished gels tend to be sticky, adhering to most surfaces except glass, porcelain, polished metals, silicone-treated paper, silicon compounds, for example. In fact, some gel compositions bond so tightly to some surfaces (such as photographic printer paper) that the bond layer is stronger than the gel itself. This allows the block to be easily joined to a variety of other surfaces for applications described below.

The handling of gel blocks or structures may scuff or obscure the gel surfaces, which can impede viewing of internal portions. However, clarity can be returned by, for example, heating the surface with a blow dryer or heat gun or wiping the surface with a light oil.

The gel can also be cut or sculpted with conventional knives (a “hot knife” is even better), or heat welded or cast it into heated molds to produce just about any conceivable shape desired.

Because it is waterproof, the gel can be easily washed off to remove dirt, hair, etc. All of these things make the gel composition very easy to store, transport and display, which is essentially impractical with ordnance gelatin (e.g. continuous refrigeration required). Similarly, standard ordnance gelatin cannot be melted or frozen, without impairing its properties. In contrast, the gel compositions described herein seems to be unaffected by such things, thereby allowing one to do more things with it.

A ballistics gel composition according to the inventive subject matter herein is sold under the name PERMA-GEL™ and is available from Perma-Gel, Inc. of Portland and Albany Oreg. The gel may be provided as part of a kit. The kit in its most basic form includes a body of gel composition or raw materials for making a body of the gel composition; and a gel heating unit, such as an oven, that allows for controlled heating of the gel composition. In certain embodiments the kit includes as the gel heating unit a conventional roaster oven, which optionally may have some modifications. For example, the oven may be modified with a temperature governor to maintain the gel composition below a predetermined temperature that may result in safety and/or performance concerns. Typically, for most gels contemplated, the temperature-limiting governor should keep the temperature below about 260° F. Attempting to use temperatures above 260° F. (even though no flammability concerns may arise until temperatures above 360° F. are attained) may cause compositions such as the PERMA-GEL™ composition, to gradually turn yellow, tacky and somewhat weaker in mechanical strength, thereby limiting its useful life. The hotter liquid may also pose a greater safety risk. Therefore, temperatures in excess of 300° F. generally should not be used. A thermometer, such as a candy thermometer should be used to determine true gel temperature.

The kit may also include one or more optional divider plates or molds for creating gel bodies of predetermined sizes and/or shapes. Optional metal channels or other connectors for securing divider plates may be provided in a heating unit. For ballistic gel blocks. the kit may include a block of gel or raw materials for making the block so as to produce a block of about 6″×11.5″×17.5″ or any other desired size.

A ballistics gel composition for use in a kit may be prepared byd melting the raw base mix as specified in steps 5-9 below. (Thereafter, follow steps 1-9 in the normal sequence.)
1) Allow finished gel blocks to come to thermal equilibrium at room temperature (55-75° F.) for at least 4 hrs before shooting. PERMA-GEL™ composition is designed to be used at room temperature, unlike ordnance gelatin which requires long cooling times (36-48 hr) at 4° C./39.2 deg F., followed by the necessity of shooting within about 20 minutes.
2) Shoot bullets into two blocks arranged end-to-end, with at least one inch between shots and at least one inch from block surfaces. Non-expanding “ball”, “Full Metal Jacket” and certain types of low-velocity (i.e., less than about 1600 fps) pistol ammunition may penetrate beyond two block lengths: always use appropriate backstops.
3) After observing, photographing, etc. begin the recycling process at a time of convenience (i.e., no limitation in shelf-life).
4) Press dry, used gel blocks back into the clean, drop inner roaster pan. This is most easily done by slicing about 1¼ inches off one end of each block, rotating the blocks 90° from their original orientation in the pan, then placing the two end slices in the gap between the blocks. A block may be used with a screen, filter, or filtration bag for remelting and separating gel from fragments. For example, a filtration bag ma)y be fashioned from a pantyhose closed with a knot and a block placed therein. The block is melted through the bag, which retains bullet fragments, etc. As noted elsewhere herein, care should be taken to ensure that no water drops or perspiration are allowed to enter the gel or pan, as this results in the formation of bubbles.
5) Set the roaster at about 225-250° F. and cover with the lid. Temperatures above about 250° F. even in localized regions, may result in slight smoking and discoloration, thereby reducing the useful life of the gel. Although not a fire-safety concern (flash point is above 360° F., which should never be approached), resist the temptation to accelerate melting by setting the oven above 250° F., as this will only shorten useful life of the gel without significantly reducing melting time. Individual oven thermostats vary somewhat, and it may therefore be helpful to use an ordinary kitchen thermometer to determine actual gel temperatures.
6) After about 3-4 hours, most or all of the gel should have melted. If not, or if there are unacceptable numbers of bubbles present, some gentle cutting and/or stirring with a paint stick or the silicone spatula provided will shorten time required for complete melting. When there is sufficient clarity, the optional divider panels mats be carefully inserted by slowly lowering it down into the hot gel, making certain that the divider engages any metal channels attached to the inside ends of the inner pan. Skin contact should be avoided. Cover with the lid. Turn power off in about one hour or when essentially all bubbles have risen to the surface.
7) Keep the lid in place throughout cooling to protect gel surfaces from dirt, insects, etc. Do not remove inner pan containing gel from oven for at least two hours.
8) Before removing gel blocks from the mold, gel should cool to a maximum temperature less than about 80° F. With natural convective cooling at normal room temperatures, this will require about 16 hr, but this time can be reduced by first allowing gel to cool in the oven for at least two hours, then removing and floating the pan in cold water or placing it in a refrigerator. With PERMA-GEL™, compositions, unlike ordnance gelatin, there are no detrimental effects from sub-freezing temperatures.
9) To help remove cooled gel blocks from the mold, pour about one cup of water on each block to serve as a lubricant between fingers and the gel. Separate gel from all vertical mold surfaces (including glass divider) with fingertips. Gently slide a wet, flattened hand between gel and mold at one end of a block, then lift upward while slipping fingertips under the block. (Caution: excessive force can tear the gel.) Similarly, remove the second block.

Gel Composition Applications Beyond Standard Ballistics Gel

Gel compositions according to the teachings herein may be used or adapted for use in a variety of applications beyond ballistics or ordnance gel that meets FBI standards. For example, it may be used to provide an enhanced ordnance gel. The forensics world seems to agree with the logic that a gelatin approved under the FBI standard does not really simulate a mammalian body very well. This is rather obvious when one considers that a body is really a complex assortment of different substances and structures, each with its own set of physical and mechanical properties. Examples are skin, bones, sinews, soft organs (lung, heart, liver, kidney, et al), as well as fluids, muscle and empty spaces. Even muscle tissue, is a fibrous material with highly directional (anisotropic) properties, and an isotropic substance like gelatin or the gel compositions described herein can never really duplicate muscle. While body parts such as bones and some other anatomical structures have been incorporated into gelatin, gelatin is not transparent enough to allow evaluation of bullet-bone interactions. Accordingly, the present inventive subject matter contemplates improved combinations and configurations of gel with simulants for bone and other anatomical structures.

The simulated structures may be formed of plastics, composites, fibers (natural and synthetic), flakes, tubular structures (vein simulant), and other materials that match or are similar to desired structures. In certain embodiments the combination of such structures with a gel results in at least one discontinuous phase (bone simulant, artery or vein simulant, flake, fiber, needle, sphere, etc.) that coexists with one or more continuous phases, the latter being a gel material. Also, discontinuous phase particles could be distributed in a gel to form families of composite materials referred to as “functionally gradient” materials.

For instance, there is high variability to the properties of human skin. From the stand point of forensic science, this is an important factor in how a bullet penetrates a body. The elasticity of skin reportedly varies greatly with different age groups, sexes, races, etc, as well from animal to animal. A skin simulant may be achieved by impregnating cloth, paper, hair, and/or other type of textile or fibrous material with gel compositions described herein to provide a layer in a gel that simulates make strong/elastic types of “skin” (or impact/penetration shielding) and found it to be quite easy to make a wide variety of sheet materials. The “,skin additives” may conveniently be added to the gel while it is in a molten condition, for example to either create a sub-layer in a monolithic block or a separate, thin layer of gel that can be bonded or attached to a monolithic block. A separate skin simulant layer has the advantage of allowing a forensics scientist or other user to customize a gel block according to desired skin types. (As used herein, the term skin simulant means skin intended to represent human skin or any other kind of animal skin or hide, with and without hair.) Similarly, a separate layer may be formed to represent different types of clothing, shielding, or other layered structure that a bullet or any other kind of projectile (including projectiles generated by explosions) that may present an initial point of impact on a person, animal or other target object.

The following are some examples of applications of the gel compositions as “bio-material”:

By molding or sculpting the gel compositions may be sized and shaped, by molding or sculpting, as specific body organs or parts, such as eyeballs, for allowing surgical instruments and devices to be used in medical tests, demonstrations and training. The gel compositions may be colored or include discontinuous phases or functional gradients to more closely simulate actual organs or other body parts.

Similarly, the SMTS compositions may be fashioned into prosthetic devices that more closely match the feel and other properties of an actual part. Here too, there may be a discontinuous phase or functional gradient for a closer match. For instance a mold (e.g. of latex) could be made of an amputee's intact leg or arm, peel it off inside-out (i.e., evert it), place it around a modern mechanical limb and fill it with a type of SMTS to make an aesthetically pleasing prosthesis for the missing limb. There are also contemplated other cosmetic and/or prosthetic uses for SMTS because it feels quite “flesh-like”. It will conform rather easily to body contours, because it tends to “creep” at temperatures above about 85° F.

Likewise the gel compositions may be used as a supplement to a natural body part, for example, as cushion used with a shoe, or a pad to protect a joint.

The SMTS gels may also be used in mechanical testing to represent a body part or organ. For example, they may be used as a simulator of the pressure a part may apply to an electronics device, such as a computer mouse, to help improve the design of such devices

In addition to biomaterials applications, the gel compositions disclosed herein are also contemplated for use in manufactured or do-it-yourself kit with which to make soft fishing lures, coated sinkers, and other fishing attachments.

They may also be fashioned for protective or comfort pads. For example, they may be used to provide custom-fitted grips or pads for such things as hand tools, golf clubs, fishing rods, etc. In these applications, a consumer could create custom grips or pads by placing the article in a moderately- heated environment (e.g., less than 140° F.), forming the grip or pad, for example, by squeezing it (perhaps with light gloves on), then allowing the article to cool and harden in the custom-fit configuration.

In another possible application, the gel compositions may be blended with a very dense (e.g., tungsten powder) particulate to make body-conformable scuba diving weights as a non-toxic substitute for existing lead-based weights.

In another possible application, the gel compositions have been found to have optical properties similar to fiber optics: a light shining on a gel composition surface is very efficiently transmitted through it around corners, etc.

All patent and non-patent literature cited herein is hereby incorporated by references as if listed in its entirety herein for all purposes.

Persons skilled in the art will recognize that man), modifications and variations are possible in the details, materials, and arrangements of the parts and actions which have been described and illustrated in order to explain the nature of this inventive concept and that such modifications and variations do not depart from the spirit and scope of the teachings and claims contained therein.

What is claimed:

1. A non-aqueous gel composition comprising thermoplastic block copolymers and an oil, the block copolymer being in a concentration that imparts to the gel composition sufficient properties to pass the FBI ordnance gel protocol when the gel composition is about 50-80° F.

2. The gel composition of claim 1 wherein the gel is substantially transparent.

3. The gel composition of claim 1 wherein the composition further includes a material or structure for simulating a body part as a discontinuous phase of the gel composition.

4. The gel composition of claim 1 wherein the block copolymer is selected from the group of polymers comprising:
(i) styrene-butadiene-styrene polymers;
(ii) styrene-isoprene-styrene polymers;
(iii) styrene-ethylene-butylene-styrene polymers;
(iv) styrene-ethylenepropylene polymers;
(v) styrene-ethylenebutylene polymers;
(vi) styrene-butadiene polymers; and
(vii) styrene-isoprene polymers.

5. The gel composition of claim 1 wherein the composition includes about 12 wt % to about 22 wt % of the block copolymer.

6. The gel composition of claim 1 wherein the copolymer comprises a styrenic block copolymer.

7. The gel composition of claim 6 wherein the styrenic block copolymer is a hydrogenated styrenic block copolymer.

8. A kit comprising a heating unit and the gel composition of claim 1 or the raw materials for the gel composition, the heating unit providing a receptacle area for melting the materials or gel composition to a predetermined temperature and forming a body of gel.

9. The kit of claim 8 wherein the heating unit is sized and shaped to provide a body of gel of at least 2″×2″ cross-section.

10. A method of making a muscle simulant comprising combining a thermoplastic block copolymer with an oil, applying heat so as to heat the mixture to about 200° F. to about 260° F. allowing the combination of materials to cool into a gel composition, the composition being formed anhydrously.

11. The method of claim 10 wherein the block copolymer is selected from the group of polymers comprising:
(i) styrene-butadiene-styrene polymers;
(ii) styrene-isoprene-styrene polymers;
(iii) styrene-ethylene-butylene-styrene polymers;
(iv) styrene-ethylenepropylene polymers;
(v) styrene-ethylenebutylene polymers;
(vi) styrene-butadiene polymers; and
(vii) styrene-isoprene polymers.

12. The method of claim 10 wherein the composition includes about 12 wt % to about 22 wt % of the block copolymer.

13. The method of claim 10 wherein the copolymer comprises a styrenic block copolymer.

14. The method of claim 12 wherein the styrenic block copolymer is a hydrogenated styrenic block copolymer.

15. The method of claim 10 further comprising allowing the gel to form under conditions that prevent the formation of appreciable bubbles.

16. The method of claim 10 further comprising adding a material or structure for simulating a body part as a discontinuous phase of the gel composition.

17. The method of claim 10 further comprising shaping the gel composition so that it is suitable for use in an FBI ordnance gel protocol.

18. The method of claim 10 further comprising shaping the gel composition so that it replicates a body part or region.

19. A method comprising providing a non-aqueous gel composition comprising a styrenic block copolymer and an oil, the gel composition adapted to pass the FBI ordnance gel protocol, and using the gel composition for ballistics testing.

20. The method of claim 19 further comprising melting the gel composition after testing and allowing it to reform as a gel in a desired shape.

21. A method comprising providing a gel composition comprising a styrenic block copolymer and an oil and forming the gel composition into a shape that replicates a body part.

22. A method comprising providing a nonaqueous gel composition comprising a styrenic block copolymer and an oil, the composition having a predetermined shape for simulating a body part, and using the shaped gel in an educational or medical training program or study unrelated to ballistics.

23. The method of claim 10 further comprising forming a discontinuous phase in the gel.

24. The method of claim 10 further comprising forming a functional gradient in the gel.

25. The method of claim 23 wherein the discontinuous phase corresponds to a body part or region.

26. The method of claim 24 wherein the functional gradient corresponds to a body part or region.

27. The gel of claim 1 wherein the gel composition is configured for use as a comfort or protective pad.

28. A gel composition comprising a thermoplastic block copolymer and an oil wherein the composition is configured for use as a comfort or protective pad; a prosthetic device, or a diving weight.

29. The composition of claim 28 further comprising a discontinuous phase present in the gel.

30. The composition of claim 28 further comprising a functional gradient in the gel.
